(19) United States
(12) Patent Application Publication
(10) Pub. No.: US 2025/0375141 A1
(43) Pub. Date: Dec. 11, 2025

(54) APPARATUS AND METHOD FOR ADAPTIVE NOISE DETECTION IN WEARABLE DEVICES

(71) Applicant: Anumana, Inc., Cambridge, MA (US)

(72) Inventors: Sairam Bade, Thelangana Suryapet (IN); Rakesh Barve, Bengaluru (IN); Shashi Kant, Bengaluru (IN); Shiva Verma, Bangalore (IN); Shayan Ghosh, Bangalore (IN)

(73) Assignee: Anumana, Inc., Cambridge, MA (US)

(21) Appl. No.: 18/736,186

(22) Filed: Jun. 6, 2024

(51) Int. Cl. A61B 5/318; A61B 5/00; A61B 5/256

(52) U.S. Cl. CPC A61B 5/318; A61B 5/256; A61B 5/7203; A61B 5/725; A61B 5/7267; A61B 5/742

(57) ABSTRACT

An apparatus and method for adaptive noise detection in wearable devices. The apparatus includes at least a physiological signal input channel configured to receive a physiological signal from a subject. The apparatus for adaptive noise detection in wearable devices further includes an adaptive noise detector communicatively connected to the at least a physiological signal input channel, wherein the adaptive noise detector further includes a signal characteristic model configured to generate a signal characteristic profile based on the physiological signal using profile training data, a signal output datapath, and a decision block.

### FIELD OF THE INVENTION

The present invention generally relates to the field of signal analysis. In particular, the present invention is directed to an apparatus and a method for adaptive noise detection in wearable devices.

### BACKGROUND

Wearable ECG devices present an economically viable support tool for caregivers engaged in continuous monitoring. However, these devices often record ECGs without the supervision of healthcare experts, resulting in recordings with inherent noise. Notably, the use of handheld devices for ECG recording encountered challenges, with failure attributed to difficulties in proper device handling. Real-time feedback on the signal quality of wearable ECG devices can be provided by comparing signal characteristics from each lead of the 12-lead ECG database.

### SUMMARY OF THE DISCLOSURE

In an aspect, an apparatus for adaptive noise detection in wearable devices includes at least a physiological signal input channel configured to receive a physiological signal from a subject. The apparatus for adaptive noise detection in wearable devices further includes an adaptive noise detector communicatively connected to the at least a physiological signal input channel, wherein the adaptive noise detector further includes a signal characteristic model configured to generate a signal characteristic profile based on the physiological signal using profile training data, wherein the profile training data comprises a plurality of physiological signals recorded on at least a reference device. The apparatus for adaptive noise detection in wearable devices further includes a signal output datapath. The apparatus for adaptive noise detection in wearable devices further includes a decision block, wherein the decision block configured to determine, based on the noise profile, that the physiological signal is within a quality tolerance; and transmit the physiological signal using the signal output datapath.

In another aspect, a method for adaptive noise detection in wearable devices includes at least a physiological signal input channel configured to receive a physiological signal from a subject. The apparatus for adaptive noise detection in wearable devices further includes an adaptive noise detector communicatively connected to the at least a physiological signal input channel, wherein the adaptive noise detector further includes a signal characteristic model configured to generate a signal characteristic profile based on the physiological signal using profile training data, wherein the profile training data comprises a plurality of physiological signals recorded on at least a reference device. The apparatus for adaptive noise detection in wearable devices further includes a signal output datapath. The apparatus for adaptive noise detection in wearable devices further includes a decision block, wherein the decision block configured to determine, based on the noise profile, that the physiological signal is within a quality tolerance; and transmit the physiological signal using the signal output datapath.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an apparatus for adaptive noise detection in wearable devices;

FIG. 2 is a block diagram of an exemplary embodiment of a system for adaptive noise detection in wearable devices by comparing signal characteristics extracted from a 12-lead ECG database;

FIG. 3 is a block diagram of an exemplary machine-learning process;

FIG. 4 is a diagram of an exemplary embodiment of a neural network;

FIG. 5 is a diagram of an exemplary embodiment of a node of a neural network;

FIG. 6 is an illustration of an exemplary embodiment of fuzzy set comparison;

FIG. 7 is a block diagram of an exemplary method for adaptive noise detection in wearable devices;

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus and methods for adaptive noise detection in wearable devices. The apparatus includes at least a physiological signal input channel configured to receive a physiological signal from a subject. The apparatus for adaptive noise detection in wearable devices further includes an adaptive noise detector communicatively connected to the at least a physiological signal input channel, wherein the adaptive noise detector further includes a signal characteristic model configured to generate a signal characteristic profile based on the physiological signal using profile training data, wherein the profile training data comprises a plurality of physiological signals recorded on at least a reference device. The apparatus for adaptive noise detection in wearable devices further includes a signal output datapath. The apparatus for adaptive noise detection in wearable devices further includes a decision block, wherein the decision block configured to determine, based on the noise profile, that the physiological signal is within a quality tolerance; and transmit the physiological signal using the signal output datapath.

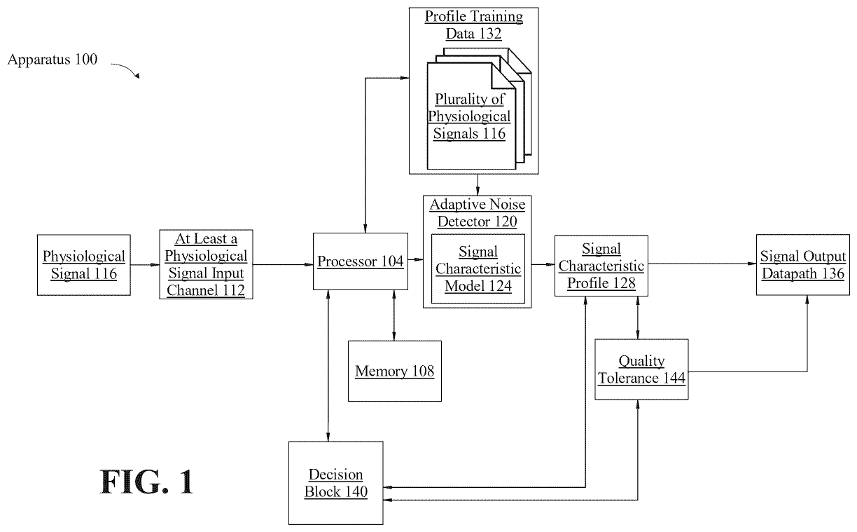

Referring now to FIG. 1, an exemplary embodiment of apparatus 100 for adaptive noise detection in wearable devices is illustrated. Apparatus 100 may include a processor 104 communicatively connected to a memory 108. As used in this disclosure, “communicatively connected” means connected by way of a connection, attachment, or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals there between may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Further referring to FIG. 1, apparatus 100 may include any “computing device” as described in this disclosure, including without limitation a microcontroller, microprocessor, digital signal processor (DSP) and/or system on a chip (SoC) as described in this disclosure. Apparatus 100 may include, be included in, and/or communicate with a mobile device such as a mobile telephone or smartphone. Apparatus 100 may include a single computing device operating independently, or may include two or more computing devices operating in concert, in parallel, sequentially or the like; two or more computing devices may be included together in a single computing device or in two or more computing devices. Apparatus 100 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Apparatus 100 may include one or more computing devices dedicated to data storage, security, distribution of traffic for load balancing, and the like. Apparatus 100 may distribute one or more computing tasks as described below across a plurality of computing devices of computing device, which may operate in parallel, in series, redundantly, or in any other manner used for distribution of tasks or memory between computing devices. Apparatus 100 may be implemented, as a non-limiting example, using a “shared nothing” architecture.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, at least a physiological signal input channel 112 is configured to receive physiological signal 116 from a subject. As used in this disclosure, a “physiological signal input channel” is a pathway that physiological signal 116 travels through. Physiological signal input channel 112 may include a wire, leads, and/or an input/port to a separate computing device, which may include a virtual input. In a non-limiting example, physiological signal input channel 112 may include a serial port along with any other system inputs and then separated according to channel of origin by a processor and/or hardware circuit. As used in this disclosure, a “physiological signal” is any information related to a subject's health that is represented in the form of a signal. This may include, without limitation, analog signals, digital signals, time-series signal data, spatial signals, frequency signals, multi-dimensional signals, and the like. In a non-limiting example, an analog signal is any continuous-time signal representing some other quantity, i.e., analogous to another quantity. For example, and without limitation, in an analog audio signal, the instantaneous signal voltage varies continuously with the pressure of the sound waves. Typically, analog signal refers to electrical signals; however, mechanical, pneumatic, hydraulic, and other systems may also convey or be considered analog signals. In another non-limiting example, a digital signal is a signal that represents data as a sequence of discrete values; at any given time it can only take on, at most, one of a finite number of values. In some cases, digital signals may represent information in discrete bands of analog levels, wherein all levels within a band of values represent the same information state. In a non-limiting example, a digital signal may be represented as a digital circuit. Typically, digital circuit signals can have two possible valid values; a binary signal or logic signal wherein the binary signal and the logic signal are represented by two voltage bands: one voltage band that is near a reference value, and the other voltage value that is near the supply voltage. The voltage bands correspond to the two values “zero” and “one” (or “false” and “true”) of the Boolean domain, wherein at any given time, a binary signal represents one binary digit (bit). Without limitation, digital signals are generally used for communications and processing within electronic devices and computer systems. In another non-limiting example, time-series signal data is information in the form of a signal that is collected and recorded over consistent intervals of time. Without limitation, time-series signal data may be used in order to extract meaningful statistics and other characteristics of the data. Time-series signal data can be classified into two main types: continuous-time series signals and discrete-time signals. Continuous-time signals are signals that are measured and recorded over a continuous range, including, but not limited to, analog signals, such as sound waves and temperature measurements (from analog devices like analog thermometers). On the other hand, discrete-time signals are recorded at specific, distinct points. For example, and without limitation, discrete-time signals may include digital sensor measurements and financial market data sampled at fixed intervals. In another non-limiting example, at least a signal may include an ECG signal wherein the ECG signal may include an ECG datum. As used herein, an “ECG datum” is a datum describing electrical activity of the heart of a subject. In some embodiments, an ECG datum may include a rhythm strip ECG datum. As used herein, a “rhythm strip ECG datum” is a datum describing electrical activity detected using a single electrode. In some embodiments, an ECG datum may include a median beat ECG datum. As used herein, a “median beat ECG datum” is a datum describing electrical activity detected using a plurality of leads and/or electrodes. In some embodiments, an ECG datum may include data collected by 2, 3, 4, 5, 6, 7, 8, 9, 10, 11, 12, or more ECG leads. For example, an ECG datum may include a median beat collected by 12 ECG leads.

With continued reference to FIG. 1, at least a physiological signal 116 may include temporal data, and metadata. As used in this disclosure, “temporal data” is information which is collected and/or recorded over a continuous-time interval or discrete-time interval. Temporal data captures signal data changes over time and provides time-stamped data recordation. As used in the current disclosure, “metadata” refers to descriptive or informational data that provides details about the digital ECG data. Metadata may include descriptive metadata, wherein descriptive metadata is configured to describe the content, context, and structure of the data. In an embodiment, metadata may include data regarding the lead system the digital ECG data was recording. ECGs are typically recorded using multiple leads, each of which provides a different view of the heart's electrical activity. Common lead systems include the 12-lead, 6-lead, 3-lead, and single-lead ECGs. The specific lead system used to generate the digital ECG data and their configurations may be documented in the metadata. In some embodiments, metadata associated with the digital ECG data may include information such as time, geographic location, medical facility names, medical professional logs, patient names, patient IDs, patient data, along with any other patient specific data. Metadata may be used to describe records of how the data has been accessed, utilized, or modified over time, aiding in understanding data usage patterns, and optimizing access.

With continued reference to FIG. 1, at least a physiological signal 116 may include electrocardiogram signals. As used in the current disclosure, a “electrocardiogram signal” is a signal representative of electrical activity of heart. The ECG signal may consist of several distinct waves and intervals, each representing a different phase of the cardiac cycle. These waves may include the P-wave, QRS complex, T wave, U wave, and the like. The P-wave may represent atrial depolarization (contraction) as the electrical impulse spreads through the atria. The QRS complex may represent ventricular depolarization (contraction) as the electrical impulse spreads through the ventricles. The QRS complex may include three waves: Q wave, R wave, and S wave. The T-wave may represent ventricular repolarization (recovery) as the ventricles prepare for the next contraction. The U-wave may sometimes be present after the T wave, it represents repolarization of the Purkinje fibers. The intervals between these waves provide information about the duration and regularity of various phases of the cardiac cycle. The ECG signal can help diagnose various heart conditions, such as arrhythmias, myocardial infarction (heart attack), conduction abnormalities, and electrolyte imbalances.

With continued reference to FIG. 1, apparatus 100 may include a transducer communicatively connected to at least a physiological signal input channel 112, wherein the transducer is configured to generate physiological signal 116. As used in this disclosure, a “transducer” is a device used to transform one kind of energy into another. When a transducer converts a quantity of energy to an electrical voltage or an electrical current it is called a sensor. A measurable quantity of energy may include sound pressure, optical intensity, magnetic field intensity, thermal pressure, etc. When a transducer converts an electrical signal into another form of energy such as sound, light, mechanical movement, it is called an actuator. It should be noted that sound is incidentally a pressure field. Actuators allow the use of feedback at the source of the measurements. In a non-limiting embodiment, at least a transducer may detect at least a cardiac phenomenon and output at least a physiological signal 116.

With continued reference to FIG. 1, a sensor may be considered as a component or with a collection of electronics such as amplifiers, decoders, filters, computer devices and at least a transducer. For the purposes of this disclosure an “instrument” is a sensor bundled with its associated electronics. However, in some embodiments, sensors may be further integrated with at least a transducer. Sensors may be integrated with wearable ECG devices such as, without limitation, ECG monitoring watches, bio stickers, portable ECG measuring devices, and the like.

With continued reference to FIG. 1, a sensor integrated with at least a transducer may be linear so that response y to a stimulus x is in the form: y(x)=Ax, 0≤x≤xₘₐₓ, A>0. It should be noted, there is a presumption that the stimulus to be positive. A is the sensitivity of the transducer gain, or the gain of the sensor. The gain is presumed to be positive for which the linear model satisfies the definition of linearity: y(x+z)=A(x+z)=y (x)+y(z). It should be noted that this example is an idealized form of a sensor and may extend beyond the linearity constraints which may include time dependency, memory, and its output keeping track of input. A more generalized sensor may include the steady state transfer function of the sensor. For this case, the sensitivity can be defined as the derivative of the output with respect to the input:

$$S = \frac{\partial y}{\partial x} .$$

In this example, the sensor exhibits sensitivities to other operating parameters (i.e., supply voltage) or temperature. For the purposes of this disclosure, “sensitivity” is the ratio of output to input. This can include electrical output and signal input or an input transducer. It can also include physical output to an electrical input, or an output transducer. Sensitivity can also be used in its usual electrical meaning. In this it would refer to a percent change of a property of a device because of a percent change in a parameter. In some embodiments this would be a percent change in gain as a result of percent change in ambient temperature. This type of sensitivity may be referred to as the Gain of a sensor.

Still referring to FIG. 1, at least a transducer with integrated sensors may not respond to arbitrarily small signals. At least a transducer may respond to signals within a specified range from zero to a sensor threshold which does not cause the output of the sensor to change. The existence of a threshold relates to the nonlinear behavior of the device and the noise. At least a transducer with an integrated sensor may fail to respond to stimuli which are arbitrarily large as well. In this case, at least a transducer integrated with a sensor may have a max range. The full range of at least a transducer integrated with a sensor may be limited by compression or clipping. Compression and clipping are results of nonlinearity and thus may include at least a transducer as a nonlinearity device.

Still referring to FIG. 1, referring to the linear equation above assuming a linear sensor is improved with the addition of a constant: y(x)=b₀+Ax. It should be noted that the equation is not linear even though it is described as a first order polynomial. The constant is called a zero offset and can be defined in two ways: a sensor reading when the input is zero, or the value of the stimulus required to make the output zero. The zero offset is corrected by subtracting b₀ from y and recovering the linear description of a sensor: y′(x)=y(x)−b₀=Ax.

With continued reference to FIG. 1, at least a transducer may include very fast measurements where it can internally store energy. At least a transducer output may depend on previous measurements the integrated sensors make. It should be noted that the sensor may exhibit memory. The time dependence of a sensor can be linear if the response is described by a linear differential equation:

$${\sum }_{n = 0}^{N} {A}_{n} \frac{{\partial }^{2} y}{\partial {t}^{n}} = {\sum }_{k = 0}^{k} {B}_{k} \frac{{\partial }^{k} x}{\partial {t}^{k}} .$$

Taking the Laplace transform of this equation:

$$y ( s , X ) = ( \frac{{\sum }_{k = 0}^{K} {B}_{k} {S}^{k}}{{\sum }_{n = 0}^{N} {A}_{n} {S}^{n}} ) x = H ( s ) X ( s ) ,$$

which is in Laplace transform space and the sensor response is still linear in stimulus x. The response of a sensor with a transfer function H(s) at time t is the convolution integral between the history of the stimulus x and the inverse Laplace transform h(t) of H(s):

$$y ( t ) = {\int }_{0}^{∞} h ( τ ) x ( t - τ ) d τ .$$

At least a transducer may behave like a low pass filter, wherein there is a delayed response to their input. There is a limit to the maximum stimulus frequency that can be detected. The maximum frequency a sensor can interpret is approximately the inverse of its response time.

Still referring to FIG. 1, apparatus 100 includes adaptive noise detector 120 communicatively connected to at least a physiological signal input channel 112. Additionally, adaptive noise detector 120 includes signal characteristic model 124 configured to generate signal characteristic profile 128 based on physiological signal 116 using profile training data 132. Profile training data 132 includes plurality of physiological signals 116 recorded on at least a reference device. As used in this disclosure, an “adaptive noise detector” is a component that monitors an input signal and identifies signal characteristics within the input signal. Adaptive noise detector 120 dynamically adjusts its parameters based on the input signal characteristics. In a non-limiting example, adaptive noise detector 120 may monitor physiologic signal 116 by receiving physiological signal 116 as input from at least a physiological signal input channel 112 and identify signal characteristic profile 128. As used in this disclosure, a “signal characteristic model” is a system that is designed to determine and classify whether the physiological signal data is within a specified distribution. In a non-limiting embodiment, signal characteristic model 124 may decide whether physiological signal 116 is within a specified distribution by comparing the signal characteristic of physiological signal 116 with profile training data 132. In another non-limiting embodiment, signal characteristic model 124 may extract signal characteristics from physiological signal 116 by providing a score that identifies the deviation between physiological signal 116 and the target or nominal signal frequency. As used in this disclosure, a “signal characteristic” is a specific attribute of a signal that can be measured, analyzed, or quantified to provide information about the signal's composition. A signal characteristic may include time-domain features, such as, without limitation, RR intervals (the time interval between successive R waves in an electrocardiogram signal), P-wave duration (the length of time measured from the beginning to the end of the P wave in an electrocardiogram signal), frequency-domain features such as, without limitation, power spectral density (the distribution of energy per unit frequency of a signal), and morphological features such as, without limitation, QRS complex shape (a feature observed in an electrocardiogram signal, representing the depolarization of the ventricles of the heart). Signal characteristic may also include advanced features which may be derived using wavelet transforms or principal component analysis (PCA) to capture more subtle aspects of an electrocardiogram signal. As previously discussed, signal characteristic model 124 may identify signal characteristic profile 128 of physiological signal 116 as high-fidelity quality signals or noisy signals. As used in this disclosure, a “signal characteristic profile” is an attribute of any signal. Without limitation, signal characteristic 128 may include patterns in the frequency domain and/or time domain. In a non-limiting embodiment, signal characteristic profile 128 may be generated by adaptive noise detector 120 as a function of signal characteristic model and physiological signal 116. In another non-limiting embodiment, signal characteristic profile 128 may be received by signal output datapath 136, as discussed in more detail below. Signal characteristic model 124 may include a system designed to analyze, identify, and extract patterns from signal data. In a non-limiting embodiment, a signal characteristic model 124 may include one or more signal processing modules. As used in this disclosure, a “signal processing module” is a component that is designed to manipulate, analyze, or transform signal data. In a non-limiting example, a signal processing module may include any combination of signal processing modules. For example, and without limitation, a signal processing module may remove noise, filter certain frequencies, enhance specific signal characteristics (as described in more detail below), compress the signal, extract certain signal features, and the like. One or more signal processing modules may be used to modify and/or manipulate the signal.

With continued reference to FIG. 1, “profile training data” is a collection of information used to train a system. In a non-limiting example, profile training data 132 may include signal characteristic profile 128 that may be associated with physiological signal 116. Without limitation, profile training data 132 may be used to train signal characteristic model 124 to detect and identify particular signal features in physiological signal 116. In a non-limiting example, profile training data may include a plurality of physiological signals 116. Plurality of physiological signals may include standard 12-lead electrocardiogram data. As used in this disclosure, “a standard 12-lead electrocardiogram database” is a database designed to store and organize a collection of standard 12-lead electrocardiogram signals. In a non-limiting example, the standard 12-lead electrocardiogram database may include a collection of organized signal data that is used to categorize and/or verify other signal data. In a non-limiting example, the standard 12-lead electrocardiogram database may include information related to signal data. In another non-limiting example, the standard 12-lead electrocardiogram database may include raw signal data and/or processed signal data. The standard 12-lead electrocardiogram database may be created using real electrocardiogram signal data from electronic health records, and the like. As used in this disclosure, a “standard 12-lead electrocardiogram signal” is a measurement the electrical activity of a heart from 12 different perspectives. A “lead,” as used in this disclosure is one or more electrodes attached to the skin to detect a heart's electric signals. In a non-limiting embodiment a standard 12-lead electrocardiogram signal may include a graphical record of the direction and magnitude of the electrical activity generated by the depolarization and repolarization of the atria and ventricles of the heart. As used in this disclosure, “a plurality of clinical transducers” is a transducer device used in the medical field to measure, analyze, and/or quantify electrical signals in a body. Plurality of clinical transducers may generate profile training data 132 for apparatus 100 and profile training data 132 may be stored in the standard 12-lead electrocardiogram database. In a non-limiting example, profile training data 132 may be stored in various lead sets, for example, 6-lead electrocardiogram database, 8-lead electrocardiogram database, and the like. As used in this disclosure, profile training data 132 may be created using publicly available databases, private databases, databases including synthetic and/or real electrocardiogram data, and the like.

With continued reference to FIG. 1, apparatus 100 may be configured to generate signal characteristic profile 128 using a frequency profile of physiological signal 116. As used in this disclosure, a “frequency profile” is a representation or description of a spectrum and/or set of component frequencies of a signal as elucidated, without limitation, by attenuation when passed through a filter such as a highpass, lowpass, and/or bandpass filter, Fourier series decomposition, and/or representation of the signal using the frequency domain, for instance as determined using a Fourier transform, Laplace transform, Z transform, or the like. In a non-limiting embodiment, a fast Fourier transform may generate the frequency profile. As used in this disclosure, a “fast Fourier transform” is an algorithm that computes the Discrete Fourier Transform (DFT) of a sequence, or its inverse (IDFT). Fourier analysis may convert a signal from its original domain (often time or space) to a representation in the frequency domain and vice versa. In a non-limiting example, the fast Fourier transform may receive a time-domain signal, such as physiological signal 116, as input and computes the discrete Fourier transform of the physiological signal 116 to represent the signal in the frequency domain wherein the representation expresses physiological signal 116 as a sum of sinusoids with different frequencies and amplitudes. Further, and without limitation, the magnitude spectrum may be obtained by converting the complex spectrum (the DFT output which represents the amplitude and phase of a specific frequency component of the signal) into its absolute value, wherein the magnitude spectrum represents the amplitude of each frequency component. The magnitude spectrum may provide a frequency profile of the signal, wherein the frequency profile represents the strength of each frequency component present in the signal, which may generate useful information about the signal's frequency content. In a non-limiting example, the frequency profile may identify specific frequencies of interest in physiological signal 116 such as, without limitation, frequencies of interest, noise in the signal, patterns in the signal, and the like.

With continued reference to FIG. 1, apparatus 100 may be configured to detect a first signal, wherein the first signal is not within a quality tolerance, and detect a second signal, wherein the second signal is within the quality tolerance. As used in this disclosure, a “signal” is a transmission of data over any communicative connection as described in this disclosure, such as an electrocardiogram signal or the like. Without limitation, the signal may include an electroencephalogram, echocardiogram, electrocardiogram, electromyogram, electrooculogram, electrodermal activity, and the like. In an embodiment, the signal may include physiological signal 116 from at least a physiological signal input channel 112. As used in this disclosure, a “quality tolerance” is a mathematical function and/or a predefined value that describes the threshold for a type of signal categorization. For instance, and without limitation, quality tolerance 144 may be a probability threshold based on a normal distribution, a uniform distribution, an exponential distribution, a binomial distribution, and the like. The threshold may, without limitation, be set by a user, for instance, in a normal distribution, the user may select to categorize a signal as high quality if the signal is within one standard deviations from the mean, otherwise the signal may be categorized as a low quality signal. In a non-limiting example, the quality tolerance may use signal characteristic profile 128 to compare physiological signal 116 and generate an output that is transmitted using signal output datapath 136.

With continued reference to FIG. 1, apparatus 100 may further include an initial signal processing module, wherein the initial signal processing module may use a 12-lead database to filter out high frequencies using a low pass filter. As used in this disclosure, an “initial signal processing module” is a first filter for the signal that extracts extraneous information from the signal. In a non-limiting example, an initial signal processing module May receive the raw input data from at least a physiological signal input channel 112. Without limitation, the raw signal data may include physiological signal 116. As used in this disclosure, a “low pass filter” is a filter that passes signals with a frequency lower than a selected cutoff frequency and attenuates signals with frequencies higher than the cutoff frequency. The exact frequency response of the filter depends on the filter design. In a non-limiting example, the low pass filter may be designed using the 12-lead database as described above. Without limitation, the low pass filter may provide a smoother form of physiological signal 116 by removing short-term signal fluctuations and leaving longer-term signal trends. In another non-limiting example, low pass filter may be implemented in an electronic circuit or a digital signal processing algorithm. Without limitation, low pass filter in an electronic circuit may include analog low pass filters using passive components (like resistors, capacitors, and inductors) or active components (like operational amplifiers) in an analog circuit. Without limitation, low pass filters in an algorithm may be implemented using digital signal processing systems or software such as, without limitation, finite impulse response (FIR) filters and/or infinite impulse response (IIR) filters.

With continued reference to FIG. 1, the initial signal processing module may include a filter, wherein the filter removes a predictable noise element. As used in this disclosure, a “filter” is a device or algorithm that modifies the characteristics of a signal by selectively passing specific frequencies through and removing other frequencies. In an embodiment, the filter may be used to initially process the physiological signal 116 and remove noise from the signal. In a non-limiting example, the filter may include a low-pass filter, a high-pass filter, a band-pass filter, and the like. As used in this disclosure, a “predictable noise element” is a type of noise interference in a signal that follows a determined pattern. In an embodiment, the predictable noise element may be removed from physiological signal 116.

With continued reference to FIG. 1, apparatus 100 is further configured to generate an error signal, transmit the error signal to a device communicatively connected to the at least a physiological signal input channel, and receive a second signal, wherein the second signal is generated using the error signal. As used in this disclosure, an “error signal” is the measured difference between an observed value and a nominal or expected value. In a non-limiting embodiment, the error signal may indicate a degree of quality of physiological signal 116. In another non-limiting example, the error signal may indicate a missing frequency characteristic in physiological signal 116.

With continued reference to FIG. 1, signal characteristic model 124 may include a statistical model, wherein the statistical model determines whether specific signal characteristics are present in at least a physiological signal 116. As used in this disclosure, a “statistical model” is a mathematical representation of relationships observed in data. A statistical model may provide insight on data regarding specific patterns and the like. A statistical model may describe, analyze, and make predictions based on the input data. A statistical model may use vectors to represent variables and/or parameters of the statistical model. As used in this disclosure, a “vector” as defined in this disclosure is a data structure that represents one or more quantitative values and/or measures the position vector. Such vector and/or embedding may include and/or represent an element of a vector space; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. A vector may be represented as an n-tuple of values, where n is one or more values, as described in further detail below; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent, for instance as measured using cosine similarity as computed using a dot product of two vectors; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute 1 as derived using a Pythagorean norm:

$$l = \sqrt{{\sum }_{i = 0}^{n} {a}_{i}^{2}} ,$$

where ai is attribute number i of the vector. Scaling and/or normalization may function to make vector comparison independent of absolute quantities of attributes, while preserving any dependency on similarity of attributes. A two-dimensional subspace of a vector space may be defined by any two orthogonal vectors contained within the vector space. Two-dimensional subspace of a vector space may be defined by any two orthogonal and/or linearly independent vectors contained within the vector space; similarly, an n-dimensional space may be defined by n vectors that are linearly independent and/or orthogonal contained within a vector space. A vector's “norm’ is a scalar value, denoted ∥a∥ indicating the vector's length or size, and may be defined, as a non-limiting example, according to a Euclidean norm for an n-dimensional vector a as:

$$‖ a ‖ = \sqrt{{\sum }_{i = 0}^{n} {a}_{i}^{2}}$$

In an embodiment, and with continued reference to FIG. 1, each frequency feature of at least a signal may be represented by a dimension of a vector space; as a non-limiting example, each element of a vector may include a number representing an enumeration of co-occurrences of first frequency feature represented by the vector with second frequency feature. As used in this disclosure, a “frequency feature” is characteristics and/or attributes of a signal that are derived from the signal's frequency component. A frequency domain feature may include information about the frequency distribution that are present in a signal. A frequency feature May describe either a static signal or a particular time period of a dynamic signal. A frequency features may be obtained from a signal by analyzing the composition of the signal frequencies to identify unique patterns and/or irregularities in the signal. Without limitation, frequency features may include power spectral density, and the like. Alternatively, or additionally, dimensions of vector space may not represent distinct frequency features, in which case elements of a vector representing a first frequency feature may have numerical values that together represent a geometrical relationship to a vector representing a second frequency feature, wherein the geometrical relationship represents and/or approximates a semantic relationship between the first frequency feature and the second frequency feature. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below.

Any vectors as described herein may be scaled, such that each vector represents each attribute along an equivalent scale of values. In an embodiment associating frequency feature to one another as described above may include computing a degree of vector similarity between a vector representing each frequency feature and a vector representing another frequency feature; vector similarity may be measured according to any norm for proximity and/or similarity of two vectors, including without limitation cosine similarity. As used in this disclosure “cosine similarity” is a measure of similarity between two-non-zero vectors of a vector space, wherein determining the similarity includes determining the cosine of the angle between the two vectors. Cosine similarity may be computed as a function of using a dot product of the two vectors divided by the lengths of the two vectors, or the dot product of two normalized vectors. For instance, and without limitation, a cosine of 0° is 1, wherein it is less than 1 for any angle in the interval (0,x) radians. Cosine similarity may be a judgment of orientation and not magnitude, wherein two vectors with the same orientation have a cosine similarity of 1, two vectors oriented at 90° relative to each other have a similarity of 0, and two vectors diametrically opposed have a similarity of −1, independent of their magnitude. As a non-limiting example, vectors may be considered similar if parallel to one another. As a further non-limiting example, vectors may be considered dissimilar if orthogonal to one another. As a further non-limiting example, vectors may be considered uncorrelated if opposite to one another. Additionally, or alternatively, degree of similarity may include any other geometric measure of distance between vectors.

A statistical model may use a matrix to represent variables and/or parameters of the statistical model. As used in this disclosure “matrix” is a rectangular array or table of numbers, symbols, expressions, vectors, and/or representations arranged in rows and columns. For instance, and without limitation, matrix may include rows and/or columns comprised of vectors representing frequency features, where each row and/or column is a vector representing a distinct frequency feature; frequency features represented by vectors in matrix may include all frequency bands over a range of frequencies as described above as the statistical model identifies the frequency features, including without limitation the magnitude and phase of a set of sinusoids at the frequency components of the signal as described above. As a non-limiting example matrix may include how a signal is distributed within different frequency bands over a range of frequencies.

Matrix may be generated by performing a singular value decomposition function. As used in this disclosure a “singular value decomposition function” is a factorization of a real and/or complex matrix that generalizes the eigen decomposition of a square normal matrix to any matrix of m rows and n columns via an extension of the polar decomposition. For example, and without limitation singular value decomposition function may decompose a first matrix, A, comprised of m rows and n columns to three other matrices, U, S, T, wherein matrix U, represents left singular vectors consisting of an orthogonal matrix of m rows and m columns, matrix S represents a singular value diagonal matrix of m rows and n columns, and matrix VT represents right singular vectors consisting of an orthogonal matrix of n rows and n columns according to the vectors consisting of an orthogonal matrix of n rows and n columns according to the function:

$${A}_{m x n} = {U}_{m x m} {S}_{m x n} {V}_{n x n}^{T}$$

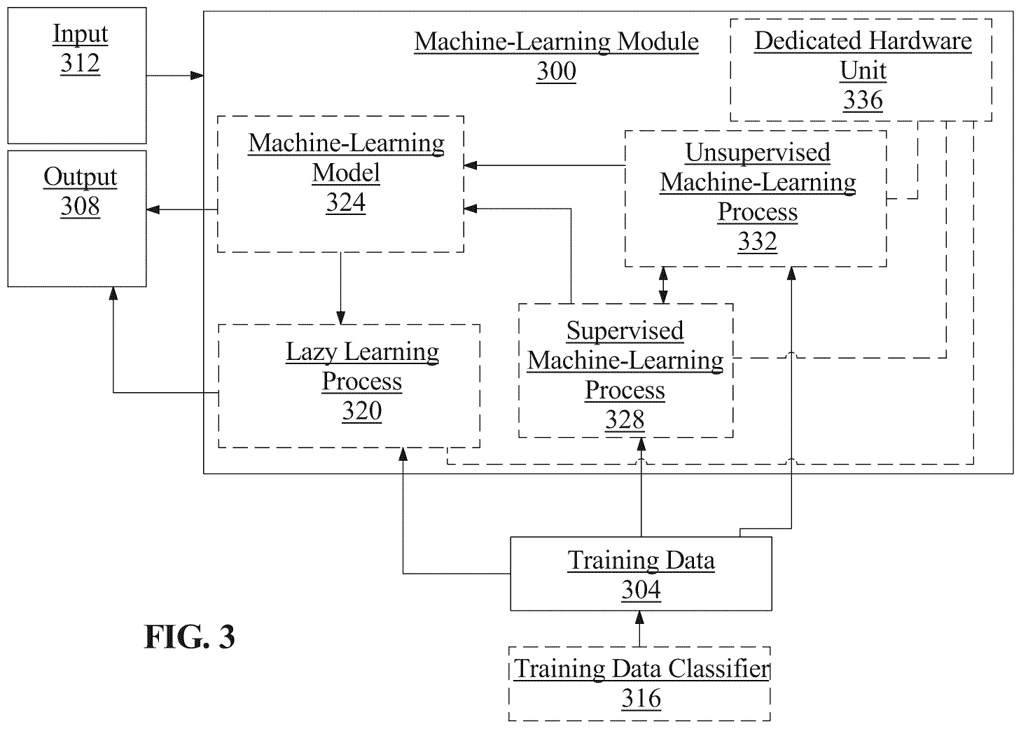

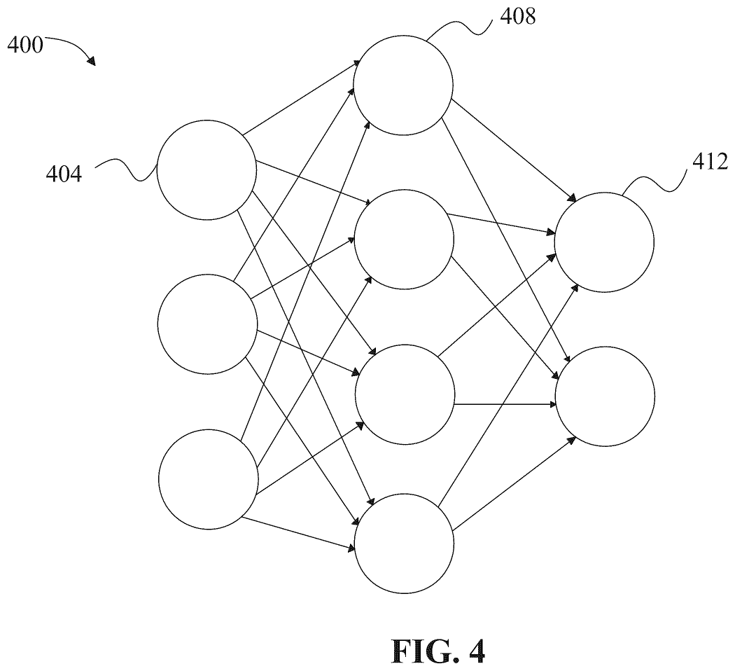

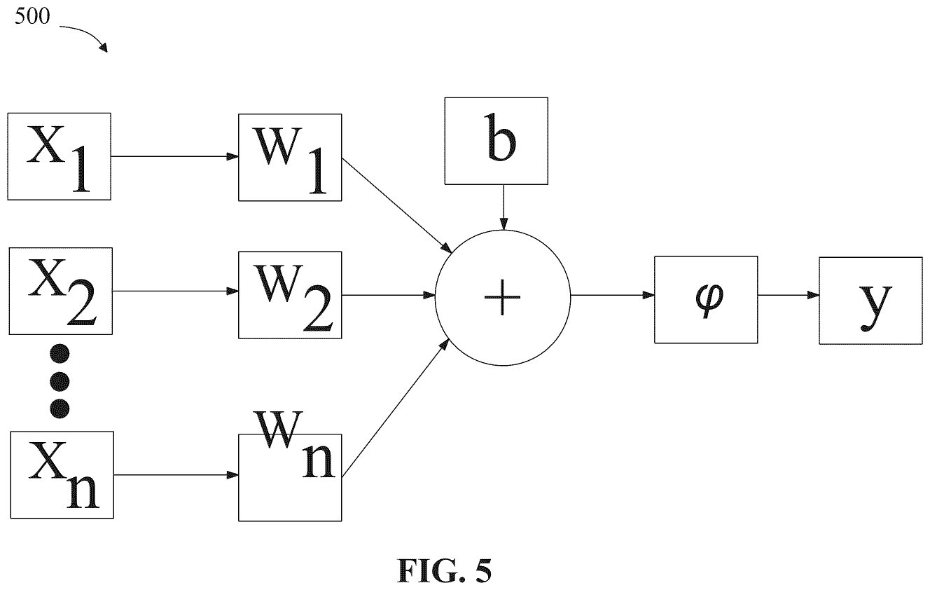

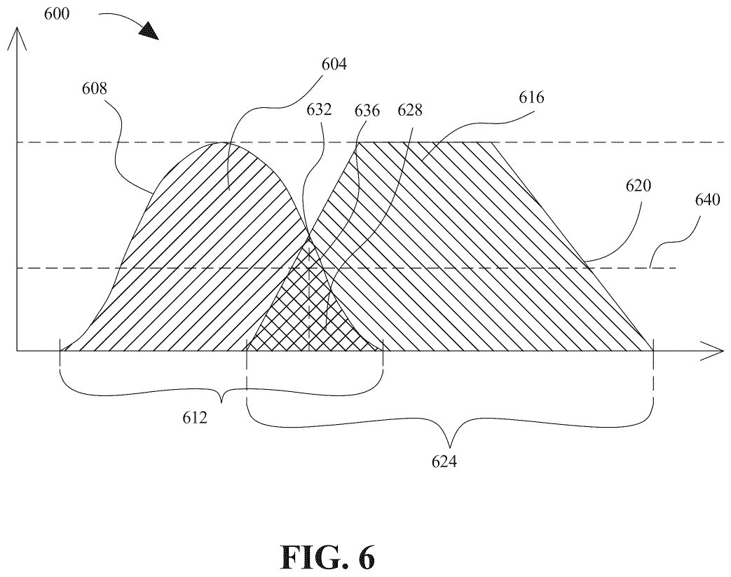

singular value decomposition function may find eigenvalues and eigenvectors of AA^{T} and A^{T}A. The eigenvectors of A^{T}A may include the columns of V^{T}, wherein the eigenvectors of AA^{T} may include the columns of U. The singular values in S may be determined as a function of the square roots of eigenvalues AA^{T} or A^{T}A, wherein the singular values are the diagonal entries of the S matrix and are arranged in descending order. Singular value decomposition may be performed such that a generalized inverse of a non-full rank matrix may be generated. A statistical model may include a fuzzy set comparison model as described in more detail in FIG. 6. Alternatively or additionally, statistical model may include a machine learning model as described in more detail in FIG. 3. Alternatively or additionally, the machine learning model as described in FIG. 3 may include a neural network as detailed in FIG. 4 and FIG. 5. The machine learning model may also include a convolutional neural network. In an embodiment, specific signal characteristics may be extracted by utilizing deep neural networks, which may learn and identify complex patterns and features in the ECG signals that may not be immediately apparent through traditional analysis methods. In some embodiments, the machine learning model may include a classifier.

With continued reference to FIG. 1, the signal characteristic model 124 may be configured to identify signal characteristics and extract signal characteristic profile 128. For instance, signal characteristic model 124 may receive 12-lead ECG signal data, determine certain attributes of the signal, and extract those attributes to store in the 12-lead ECG database to reference in later processes. In another non-limiting example, signal characteristic model 124 may use signal characteristics from the 12-lead ECG signal database to compare and identify the signal characteristic from physiological signal 116. Alternatively or additionally, signal characteristic model 124 may be configured to generate simulated 12-lead ECG signal data to further learn to identify and extract signal characteristic profile 128.

With continued reference to FIG. 1, the signal characteristic model 124 may include a neural network. Signal characteristic model 124 may use a deep neural network to compare signal data to the signal characteristics extracted from each lead of the 12-lead ECG database. In an embodiment, the neural network includes the details described in FIG. 4.

Still referring to FIG. 1, apparatus 100 further includes signal output datapath 136. As used in this disclosure, a “signal output datapath” is a channel that receives an output signal. Signal output datapath may receive, without limitation, processed physiological signal 116. Without limitation, signal output datapath 136 may include formatting and effectively delivering output information to a subject. Without limitation, signal output datapath may be displayed to a user through a graphical user interface wherein the user may interact with the output signal data.

With continued reference to FIG. 1, signal output datapath 136 may further include a display device and the computing device is further configured to conditionally display, using the display device, a notification as a function of signal characteristic profile 128. As used in this disclosure, a “display device” refers to an electronic device that visually presents information to the entity. In some cases, display device may be configured to project or show visual content generated by computers, video devices, or other electronic mechanisms. In some cases, the display device may include a liquid crystal display (LCD), a cathode ray tube (CRT), a plasma display, a light emitting diode (LED) display, and any combinations thereof. In a non-limiting example, one or more display devices may vary in size, resolution, technology, and functionality. The display device may be able to show any data elements and/or visual elements as listed above in various formats such as, textural, graphical, video among others, in either monochrome or color. The display device may include, but is not limited to, a smartphone, tablet, laptop, monitor, tablet, and the like. The display device may include a separate device that includes a transparent screen configured to display computer generated images and/or information. In some cases, the display device may be configured to present a graphical user interface (GUI) to a user, wherein a user may interact with a GUI. In some cases, a user may view a GUI through display. Additionally, or alternatively, processor 104 be connected to the display device. In one or more embodiments, transmitting feedback may include displaying feedback at the display device using a visual interface. As used in this disclosure, a “notification” is an alert of some kind delivered to an entity. In a non-limiting example a notification may be transmitted to an entity in the form of an image, graphic, text, audio, vibration, and the like. In a non-limiting example, a notification may be delivered in real-time to the display device, a client device, any kind of computing device, and the like. Without limitation, notification may provide information regarding ECG data, specifically, whether the ECG data signal characteristic was classified as of proper quality, aligned and within the distribution observed in the 12-lead ECG database, or not within the distribution observed in the 12-lead ECG database.

With continued reference to FIG. 1, the notification may include a visual element. As used in this disclosure, a “visual element” is any individual component that expresses an idea and/or conveys a message. A visual element may include visual data such as, but not limited to, images, colors, shapes, lines, arrows, icons, photographs, infographics, text, any combinations thereof, and the like. A visual element may include any data transmitted to the display device, client device, and/or graphical user interface. In some embodiments, visual element may be interacted with. For example, visual element may include an interface, such as a button or menu. In some embodiments, visual element may be interacted with using a user device such as a smartphone, tablet, smartwatch, or computer.

With continued reference to FIG. 1, displaying the notification may include a data structure. As used in this disclosure, a “data structure” is a way of organizing data represented in a specialized format on a computer configured such that the information can be effectively presented in a graphical user interface. In some cases, the data structure includes any input data. In some cases, the data structure contains data and/or rules used to visualize the graphical elements within a graphical user interface. In some cases, the data structure may include any data described in this disclosure. In some cases, the data structure may be configured to modify the graphical user interface, wherein data within the data structure may be represented visually by the graphical user interface. In some cases, the data structure may be continuously modified and/or updated by processor 104, wherein elements within graphical user interface may be modified as a result. In some cases, processor 104 may be configured to transmit to the display device the data structure. Transmitting may include, and without limitation, transmitting using a wired or wireless connection, direct, or indirect, and between two or more components, circuits, devices, systems, and the like, which allows for reception and/or transmittance of data and/or signal(s) therebetween. Data and/or signals there between may include, without limitation, electrical, electromagnetic, magnetic, video, audio, radio, and microwave data and/or signals, combinations thereof, and the like, among others. Processor 104 may transmit the data described above to a database wherein the data may be accessed from the database. Processor 104 may further transmit the data above to the display device, client device, or another computing device. As used in this disclosure, “client device” is a device that accesses and interacts with apparatus 100. For instance, and without limitation, client device may include a remote device and/or apparatus 100. In a non-limiting embodiment, client device may be consistent with a computing device as described in the entirety of this disclosure.

Still referring to FIG. 1, apparatus 100 includes decision block 140, wherein decision block 140 is configured to determine, based on signal characteristic profile 128, that physiological signal 116 is within quality tolerance 144 and transmit physiological signal 116 using signal output datapath 136. As used in this disclosure, a “decision block” is a component in a system that evaluates one or more conditions of the system output. In a nonlimiting example, decision block 140 may include one or more algorithms to control the flow of information, particularly signal characteristic profile 128 in apparatus 100. Without limitation, decision block 140 may conditionally input signal characteristic profile 128 into a downstream process as a function of quality tolerance 144. As used in this disclosure, a “downstream process” is a step or sequence of steps that occur subsequent to a first step or sequence of steps. In a non-limiting example, a downstream process may include re-capturing a second set of data from the ECG wearable device when decision block 140 determines that the first set of data from the ECG wearable device is not within quality tolerance 144. In another non-limiting example, an additional or alternative downstream process may include accepting the first set of data from the ECG and transmitting a notification to the subject that relays the quality of the first set of data collected from the ECG wearable device is within quality tolerance 144.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

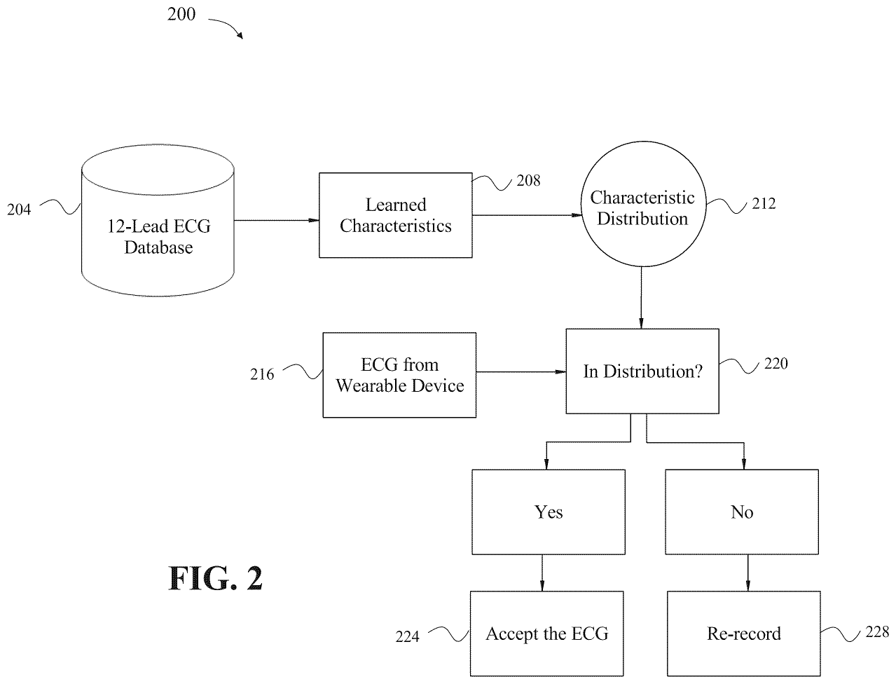

Referring now to FIG. 2, an exemplary embodiment of a system, 200, for providing real-time quality feedback for wearable ECG devices by comparing signal characteristics extracted from a 12-lead ECG database. In one or more embodiments, 12-lead ECG database, 204, is used to train a model on signal characteristics, wherein the model understands learned characteristics 208. In one or more embodiments, learned characteristics 208 is input into a model that classifies the characteristic distribution, 212. In one or more embodiments, ECG from wearable device 216 is compared to characteristic distribution 212 and another model determines whether that ECG given signal is in distribution 220. In one or more embodiments, if ECG from wearable device 216 is in distribution 220, then system 200 accepts the ECG 224. In one or more embodiments, if ECG from wearable device 216 is not in distribution 220, then system 200 re-records 228.

Referring now to FIG. 3, an exemplary embodiment of a machine-learning module 300 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm instantiated in hardware or software logic, data structures, and/or functions that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure. As a non-limiting illustrative example inputs may include raw ECG data from a transducer and outputs may include extracted and organized ECG lead data.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 300 may generate a classifier using a classification algorithm, defined as a processes whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 316 may classify elements of training data as quality ECG signal data or noisy ECG signal data.

Still referring to FIG. 3, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 3, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 3, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 3, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 3, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 3, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 3, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 3, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 3, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 3, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 3, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{new} = \frac{X - {X}_{m i n}}{{X}_{m ax} - {X}_{m i n}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{m ax} - {X}_{m i n}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons 33 skilled in the art, upon reviewing the entirety of this disclosure, will be aware of various alternative or additional approaches that may be used for feature scaling.

Further referring to FIG. 3, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, and/or generation of modified copies of existing entries. Augmentation may include performing one or more transformations on data, such as geometric, color space, affine, brightness, cropping, and/or contrast transformations of images.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 3, machine-learning processes as described in this disclosure may be used to generate machine-learning models 324. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include raw ECG signal data as described above as inputs, signal characteristics of ECG data as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

With further reference to FIG. 3, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 3, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 332 may not require a response variable; unsupervised processes 332 may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of lincar regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of cach coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the clastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g., a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 3, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 3, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 3, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 3, one or more processes or algorithms described above may be performed by at least a dedicated hardware unit 336. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 336 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 336 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 336 to perform one or more operations described herein, such as evaluation of model and/or algorithm outputs, one-time or iterative updates to parameters, coefficients, weights, and/or biases, and/or any other operations such as vector and/or matrix operations as described in this disclosure.

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

Referring now to FIG. 5, an exemplary embodiment of a node 500 of a neural network is illustrated. A node may include, without limitation, a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x) =tanh²(x), a rectified linear unit function such as f(x)=max (0, x), a “leaky” and/or “parametric” rectified linear unit function such as f(x)=max (ax, x) for some a, an exponential linear units function such as

$$f ( x ) = \{ \begin{matrix}x for x \geq 0 \\ α ( {e}^{x} - 1 ) for x < 0\end{matrix}$$

for some value of a (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh (\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs x; that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function, φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

Referring now to FIG. 6, an exemplary embodiment of fuzzy set comparison 600 is illustrated. A first fuzzy set 604 may be represented, without limitation, according to a first membership function 608 representing a probability that an input falling on a first range of values 612 is a member of the first fuzzy set 604, where the first membership function 608 has values on a range of probabilities such as without limitation the interval [0,1], and an arca beneath the first membership function 608 may represent a set of values within first fuzzy set 604. Although first range of values 612 is illustrated for clarity in this exemplary depiction as a range on a single number line or axis, first range of values 612 may be defined on two or more dimensions, representing, for instance, a Cartesian product between a plurality of ranges, curves, axes, spaces, dimensions, or the like. First membership function 608 may include any suitable function mapping first range 612 to a probability interval, including without limitation a triangular function defined by two linear elements such as line segments or planes that intersect at or below the top of the probability interval. As a non-limiting example, triangular membership function may be defined as:

$$y ( x , a , b , c ) = \{ \begin{matrix}0 , for x > c and x < a \\ \frac{x - a}{b - a} , for a \leq x < b \\ \frac{c - x}{c - b} , if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

Still referring to FIG. 6, first fuzzy set 604 may represent any value or combination of values as described above, including output from one or more machine-learning models, training data, and a predetermined class, such as, without limitation, high-fidelity signal vs noisy signals. A second fuzzy set 616, which may represent any value which may be represented by first fuzzy set 604, may be defined by a second membership function 620 on a second range 624; second range 624 may be identical and/or overlap with first range 612 and/or may be combined with first range via Cartesian product or the like to generate a mapping permitting evaluation overlap of first fuzzy set 604 and second fuzzy set 616. Where first fuzzy set 604 and second fuzzy set 616 have a region 628 that overlaps, first membership function 608 and second membership function 620 may intersect at a point 632 representing a probability, as defined on probability interval, of a match between first fuzzy set 604 and second fuzzy set 616.

Alternatively or additionally, a single value of first and/or second fuzzy set may be located at a locus 636 on first range 612 and/or second range 624, where a probability of membership may be taken by evaluation of first membership function 608 and/or second membership function 620 at that range point. A probability at 628 and/or 632 may be compared to a threshold 640 to determine whether a positive match is indicated. Threshold 640 may, in a non-limiting example, represent a degree of match between first fuzzy set 604 and second fuzzy set 616, and/or single values therein with each other or with either set, which is sufficient for purposes of the matching process; for instance, threshold may indicate a sufficient degree of overlap between an output from one or more machine-learning models and/or training data and a predetermined class, such as without limitation, high-fidelity signals or noisy signal categorization, for combination to occur as described above. Alternatively or additionally, each threshold may be tuned by a machine-learning and/or statistical process, for instance and without limitation as described in further detail below.

Further referring to FIG. 6, in an embodiment, a degree of match between fuzzy sets may be used to classify the physiological signal with the profile training data. For instance, the physiological signal has a fuzzy set matching the profile training data fuzzy set by having a degree of overlap exceeding a threshold, computing device may classify the physiological signal as belonging to the profile training data categorization. Where multiple fuzzy matches are performed, degrees of match for each respective fuzzy set may be computed and aggregated through, for instance, addition, averaging, or the like, to determine an overall degree of match.

Still referring to FIG. 6, in an embodiment, the physiological signal may be compared to multiple profile training data categorization fuzzy sets. For instance, the physiological signal may be represented by a fuzzy set that is compared to each of the multiple profile training data categorization fuzzy sets; and a degree of overlap exceeding a threshold between the physiological signal fuzzy set and any of the multiple profile training data categorization fuzzy sets may cause computing device to classify the physiological signal as belonging to profile training data categorization. For instance, in one embodiment there may be two profile training data categorization fuzzy sets, representing respectively ECG data signals within a specified distribution related to the fidelity of the signal and ECG data signals not within that specified distribution. First ECG data signals within a specified distribution categorization may have a first fuzzy set; Second ECG data signals not within a specified distribution categorization may have a second fuzzy set; and the profile training data may have ECG data signals within a specified distribution fuzzy set. Computing device, for example, may compare a training data fuzzy set with each of signal characteristic categorization fuzzy set and in signal characteristic categorization fuzzy set, as described above, and classify a training data to either, both, or neither of signal characteristic categorization nor in signal characteristic categorization. Machine-learning methods as described throughout may, in a non-limiting example, generate coefficients used in fuzzy set equations as described above, such as without limitation x, c, and o of a Gaussian set as described above, as outputs of machine-learning methods. Likewise, training data may be used indirectly to determine a fuzzy set, as training data fuzzy set may be derived from outputs of one or more machine-learning models that take the training data directly or indirectly as inputs.

Still referring to FIG. 6, a computing device may use a logic comparison program, such as, but not limited to, a fuzzy logic model to determine a signal characteristic response. An signal characteristic response may include, but is not limited to, high-fidelity signals, noisy signals, and the like; each such signal characteristic response may be represented as a value for a linguistic variable representing signal characteristic response or in other words a fuzzy set as described above that corresponds to a degree of signal quality as calculated using any statistical, machine-learning, or other method that may occur to a person skilled in the art upon reviewing the entirety of this disclosure. In other words, a given element of training data may have a first non-zero value for membership in a first linguistic variable value such as “high-fidelity” and a second non-zero value for membership in a second linguistic variable value such as “noisy” In some embodiments, determining a signal characteristic categorization may include using a linear regression model. A linear regression model may include a machine learning model. A linear regression model may be configured to map data of training data, such as degree of signal quality to one or more signal characteristic parameters. A linear regression model may be trained using a machine learning process. A linear regression model may map statistics such as, but not limited to, quality of training data. In some embodiments, determining a signal characteristic of training data may include using a signal characteristic classification model. A signal characteristic classification model may be configured to input collected data and cluster data to a centroid based on, but not limited to, frequency of appearance, linguistic indicators of quality, and the like. Centroids may include scores assigned to them such that quality of the physiological signal of profile training data may each be assigned a score. In some embodiments signal characteristic classification model may include a K-means clustering model. In some embodiments, signal characteristic classification model may include a particle swarm optimization model. In some embodiments, determining the signal characteristic of a training data may include using a fuzzy inference engine. A fuzzy inference engine may be configured to map one or more training data elements using fuzzy logic. In some embodiments, training data may be arranged by a logic comparison program into signal characteristic arrangement. A “signal characteristic arrangement” as used in this disclosure is any grouping of objects and/or data based on skill level and/or output score. This step may be implemented as described above in FIGS. 1-5. Membership function coefficients and/or constants as described above may be tuned according to classification and/or clustering algorithms. For instance, and without limitation, a clustering algorithm may determine a Gaussian or other distribution of questions about a centroid corresponding to a given signal quality level, and an iterative or other method may be used to find a membership function, for any membership function type as described above, that minimizes an average error from the statistically determined distribution, such that, for instance, a triangular or Gaussian membership function about a centroid representing a center of the distribution that most closely matches the distribution. Error functions to be minimized, and/or methods of minimization, may be performed without limitation according to any error function and/or error function minimization process and/or method as described in this disclosure.

Further referring to FIG. 6, an inference engine may be implemented according to input and/or output membership functions and/or linguistic variables. For instance, a first linguistic variable may represent a first measurable value pertaining to training data, such as a degree of quality of a signal, while a second membership function may indicate a degree of in signal characteristic of a subject thereof, or another measurable value pertaining to training data. Continuing the example, an output linguistic variable may represent, without limitation, a score value. An inference engine may combine rules. The degree to which a given input function membership matches a given rule may be determined by a triangular norm or “T-norm” of the rule or output membership function with the input membership function, such as min (a, b), product of a and b, drastic product of a and b, Hamacher product of a and b, or the like, satisfying the rules of commutativity (T(a, b)=T(b, a)), monotonicity: (T(a, b)≤T(c, d) if a≤c and b≤d), (associativity: T(a, T(b, c))=T(T(a, b), c)), and the requirement that the number 1 acts as an identity element. Combinations of rules (“and” or “or” combination of rule membership determinations) may be performed using any T-conorm, as represented by an inverted T symbol or “1,” such as max (a, b), probabilistic sum of a and b (a+b−a*b), bounded sum, and/or drastic T-conorm; any T-conorm may be used that satisfies the properties of commutativity: ⊥(a, b)=⊥(b, a), monotonicity: ⊥(a, b)≤⊥(c, d) if a≤c and b≤d, associativity: ⊥(a, ⊥(b, c))=⊥(⊥(a, b), c), and identity element of 0. Alternatively or additionally T-conorm may be approximated by sum, as in a “product-sum” inference engine in which T-norm is product and T-conorm is sum. A final output score or other fuzzy inference output may be determined from an output membership function as described above using any suitable defuzzification process, including without limitation Mean of Max defuzzification, Centroid of Area/Center of Gravity defuzzification, Center Average defuzzification, Bisector of Arca defuzzification, or the like. Alternatively or additionally, output rules may be replaced with functions according to the Takagi-Sugeno-King (TSK) fuzzy model.

Further referring to FIG. 6, training data to be used may be selected by user selection, and/or by selecti on of a distribution of output scores. Each signal characteristic categorization may be selected using an additional function such as insignal characteristic as described above.

Still referring to FIG. 6, a computing device may use a logic comparison program, such as, but not limited to, a fuzzy logic model to determine a risk level response. A risk level response may include, but is not limited to, high risk, moderate risk, low risk, and the like; each such risk level response may be represented as a value for a linguistic variable representing risk level response or in other words a fuzzy set as described above that corresponds to a degree of risk as calculated using any statistical, machine-learning, or other method that may occur to a person skilled in the art upon reviewing the entirety of this disclosure. In other words, a given element of input may have a first non-zero value for membership in a first linguistic variable value such as “high risk” and a second non-zero value for membership in a second linguistic variable value such as “moderate risk” In some embodiments, determining a first categorization may include using a linear regression model. A linear regression model may include a machine learning model. A linear regression model may be configured to map data of input such as degree of risk to one or more category parameters. A linear regression model may be trained using a machine learning process. A linear regression model may map statistics such as, but not limited to, quality of input risk. In some embodiments, determining a category of input may include using a risk classification model. A risk classification model may be configured to input collected data and cluster data to a centroid based on, but not limited to, frequency of appearance, linguistic indicators of quality, and the like. Centroids may include scores assigned to them such that quality of risk of an input may each be assigned a score. In some embodiments risk classification model may include a K-means clustering model. In some embodiments, risk classification model may include a particle swarm optimization model. In some embodiments, determining the risk of an input may include using a fuzzy inference engine. A fuzzy inference engine may be configured to map one or more input data elements using fuzzy logic. In some embodiments, input may be arranged by a logic comparison program into risk arrangement. A “risk arrangement” as used in this disclosure is any grouping of input and/or data based on skill level and/or output score. This step may be implemented as described above in FIGS. 1-5. Membership function coefficients and/or constants as described above may be tuned according to classification and/or clustering algorithms. For instance, and without limitation, a clustering algorithm may determine a Gaussian or other distribution of questions about a centroid corresponding to a given risk level, and an iterative or other method may be used to find a membership function, for any membership function type as described above, that minimizes an average error from the statistically determined distribution, such that, for instance, a triangular or Gaussian membership function about a centroid representing a center of the distribution that most closely matches the distribution. Error functions to be minimized, and/or methods of minimization, may be performed without limitation according to any error function and/or error function minimization process and/or method as described in this disclosure.

Further referring to FIG. 6, an inference engine may be implemented according to input and/or output membership functions and/or linguistic variables. For instance, a first linguistic variable may represent a first measurable value pertaining to input such as a degree of risk of an element, while a second membership function may indicate a degree of in risk of a subject thereof, or another measurable value pertaining to an input. Continuing the example, an output linguistic variable may represent, without limitation, a score value. An inference engine may combine rules, such as: “if the risk level is ‘high’ and the popularity level is ‘high’, the question score is ‘high’”—the degree to which a given input function membership matches a given rule may be determined by a triangular norm or “T-norm” of the rule or output membership function with the input membership function, such as min (a, b), product of a and b, drastic product of a and b, Hamacher product of a and b, or the like, satisfying the rules of commutativity (T(a, b)=T (b, a)), monotonicity: (T(a, b)≤T (c, d) if a≤c and b≤d), (associativity: T(a, T(b, c))=T(T (a, b), c)), and the requirement that the number 1 acts as an identity element. Combinations of rules (“and” or “or” combination of rule membership determinations) may be performed using any T-conorm, as represented by an inverted T symbol or “1,” such as max (a, b), probabilistic sum of a and b (a+b−a*b), bounded sum, and/or drastic T-conorm; any T-conorm may be used that satisfies the properties of commutativity: ⊥(a, b)=⊥(b, a), monotonicity: ⊥(a, b)≤⊥(c, d) if a≤c and b≤d, associativity: ⊥(a, ⊥(b, c))=⊥(⊥(a, b), c), and identity element of 0. Alternatively or additionally T-conorm may be approximated by sum, as in a “product-sum” inference engine in which T-norm is product and T-conorm is sum. A final output score or other fuzzy inference output may be determined from an output membership function as described above using any suitable defuzzification process, including without limitation Mean of Max defuzzification, Centroid of Arca/Center of Gravity defuzzification, Center Average defuzzification, Bisector of Area defuzzification, or the like. Alternatively or additionally, output rules may be replaced with functions according to the Takagi-Sugeno-King (TSK) fuzzy model.

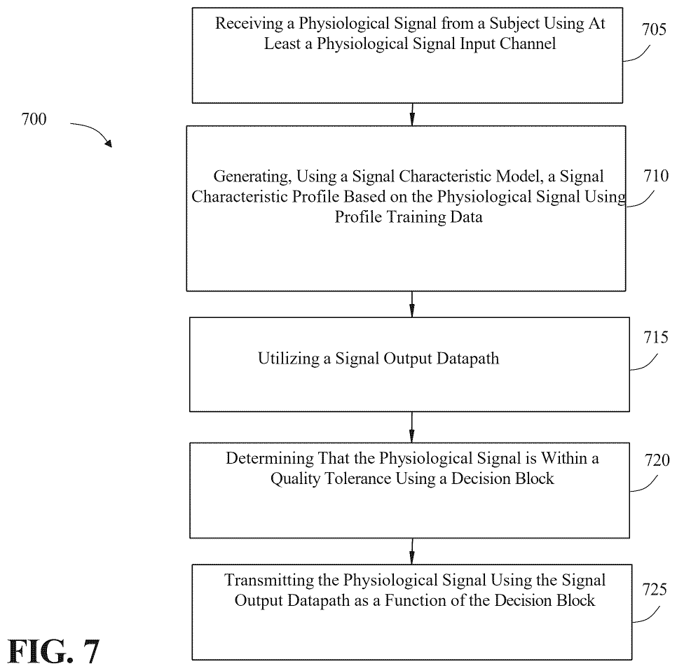

Referring now to FIG. 7, a flow diagram of an exemplary method 700 for adaptive noise detection in wearable devices is illustrated. At step 705, method 700 includes receiving, using at least a physiological signal input channel, a physiological signal from a subject. This may be implemented as described and with reference to FIGS. 1-6.

Still referring to FIG. 7, at step 710, method 700 includes generating, using a signal characteristic model, a signal characteristic profile based on the physiological signal using profile training data. This may be implemented as described and with reference to FIGS. 1-6.

Still referring to FIG. 7, at step 715, method 700 includes utilizing a signal output datapath. This may be implemented as described and with reference to FIGS. 1-6.

Still referring to FIG. 7, at step 720, method 700 includes determining, using a decision block, that the physiological signal is within a quality tolerance. This may be implemented as described and with reference to FIGS. 1-6.

Still referring to FIG. 7, at step 725, method 700 includes transmitting, using the decision block, the physiological signal using the signal output datapath. This may be implemented as described and with reference to FIGS. 1-6.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

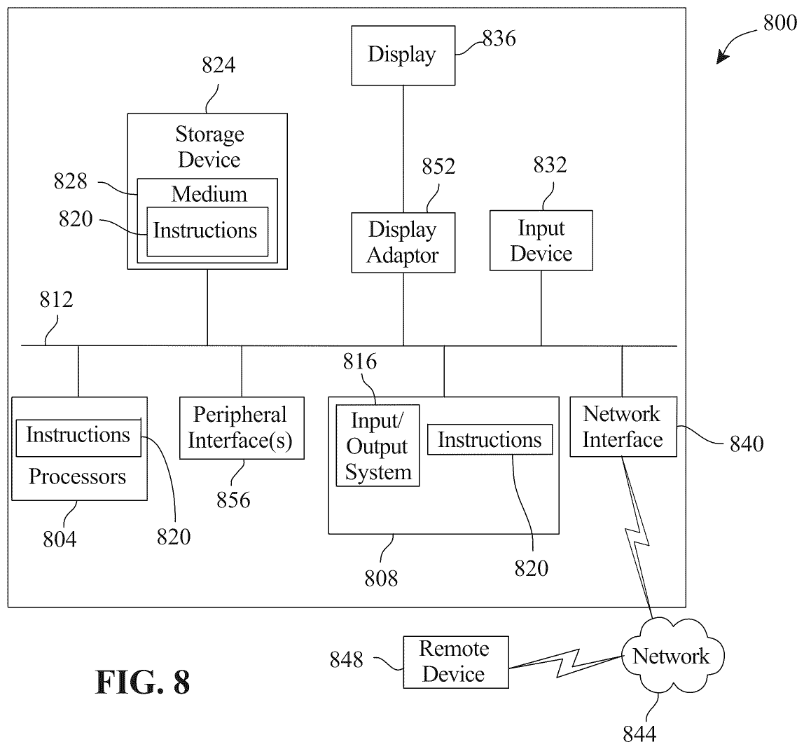

FIG. 8 shows a diagrammatic representation of one embodiment of computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 cmbodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 924 may be connected to bus 812 by an appropriate interface (not shown). Example interfaces include, but are not limited to, SCSI, advanced technology attachment (ATA), serial ATA, universal serial bus (USB), IEEE 1394(FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for adaptive noise detection in wearable devices,
wherein the apparatus comprises:
a sensor, wherein the sensor is configured to receive a physiological signal from a subject;
at least a processor;
a memory communicatively connected to the at least a processor, wherein the memory contains instructions configuring the at least a processor to receive the physiological signal and a profile training data set, wherein the profile training data set comprises outputs correlated to inputs, wherein the inputs comprise a plurality of physiological signal data and the outputs comprise a plurality of signal characteristic profile data;
at least a physiological signal input channel configured to receive the physiological signal;
a dedicated hardware unit, communicatively connected to the at least a processor, configured to sanitize the profile training data set, wherein the dedicated hardware unit comprises circuitry configured to perform signal processing operations, wherein sanitizing the profile training data set comprises:
determining by the dedicated hardware unit that at least one training data entry of the profile training data set has a signal to noise ratio below a threshold value; and
removing the at least one training data entry from the profile training data set to create a sanitized profile training data set;
an adaptive noise detector communicatively connected to the at least a physiological signal input channel, wherein the adaptive noise detector comprises a signal characteristic model which is configured to:
receive the sanitized profile training data set
train, iteratively, the signal characteristic model using the sanitized profile training data set, wherein training the signal characteristic model includes retraining the signal characteristic model with previous results of the signal characteristic model; and
generate a signal characteristic profile as a function of the physiological signal using the trained signal characteristic model;
a signal output datapath, wherein the signal output datapath receives the signal characteristic profile and is displayed through a graphical user interface, wherein a user may interact with the signal characteristic profile; and
a decision block operably connected to the at least a processor, wherein the decision block in combination with the at least a processor is configured to:
determine, based on the signal characteristic profile, that the physiological signal is within a quality tolerance, wherein the quality tolerance is a probability threshold based on a distribution of a plurality of physiological signals; and
transmit a result of whether the physiological signal is within the quality tolerance using the signal output datapath.

2. The apparatus of claim 1, wherein the sensor is communicatively connected to the at least a physiological signal input channel.

3. The apparatus of claim 1, the apparatus is further configured to determine a frequency profile and then generate the signal characteristic profile using the frequency profile of the physiological signal.

4. The apparatus of claim 1, the apparatus is further configured to:
detect a first signal from the at least a physiological signal input channel, wherein the first signal is not within the quality tolerance;
detect a second signal from the at least a physiological signal input channel, wherein the second signal is within the quality tolerance.

5. The apparatus of claim 1, wherein the apparatus further comprises an initial signal processing module and a 12-lead database to filter high frequencies from the physiological signal using a low pass filter.

6. The apparatus of claim 5, wherein the low pass filter removes a predictable noise element.

7. (canceled)

8. The apparatus of claim 1, wherein the signal characteristic model comprises a statistical model.

9. The apparatus of claim 8, wherein the statistical model comprises a machine learning model.

10. The apparatus of claim 1, wherein the at least a processor is configured to conditionally display, using the graphical user interface, a notification as a function of the signal characteristic profile.

11. A method for adaptive noise detection in wearable devices, wherein the method comprises:
receiving, using a sensor, a physiological signal from a subject;
receiving, using a processor, the physiological signal and a profile training data set, wherein the profile training data set comprises outputs correlated to inputs, wherein the inputs comprise a plurality of physiological signal data and the outputs comprise a plurality of signal characteristic profile data;
sanitizing, using a dedicated hardware unit communicatively connected to the processor, the profile training data set, wherein the dedicated hardware unit comprises circuitry configured to perform signal processing operations, wherein sanitizing the profile training data set comprises:
determining by the dedicated hardware unit that at least one training data entry of the profile training data set has a signal to noise ratio below a threshold value; and
removing the at least one training data entry from the profile training data set to create a sanitized profile training data set;
receiving, using at least a physiological signal input channel, the physiological signal from the subject;
generating, using an adaptive noise detector, a signal characteristic profile based on the physiological signal, wherein the adaptive noise detector comprises a signal characteristic model, wherein generating the signal characteristic profile comprises:
receiving the sanitized profile training data set;
training, iteratively, the signal characteristic model using the sanitized profile training data set, wherein training the signal characteristic model includes retraining the signal characteristic model with previous results of the signal characteristic model; and
generating a signal characteristic profile as a function of the physiological signal using the trained signal characteristic model;
receiving, at a signal output datapath, the signal characteristic profile;
displaying, through a graphical user interface, the signal output datapath, wherein a user may interact with the signal characteristic profile;
determining, using a decision block communicatively connected to the processor, that the physiological signal is within a quality tolerance, wherein the quality tolerance is a probability threshold based on a distribution of a plurality of physiological signals; and
transmitting, using the signal output datapath, a result of whether the physiological signal is within the quality tolerance.

12. The method of claim 11, further comprising communicatively connecting the sensor to the at least a physiological signal input channel, the physiological signal.

13. The method of claim 11, further comprising determining a frequency profile and then generating the signal characteristic profile using the frequency profile of the physiological signal.

14. The method of claim 11, further comprising:
detecting a first signal from the at least a physiological signal input channel, wherein the first signal is not within the quality tolerance;
detecting a second signal from the at least a physiological signal input channel, wherein the second signal is within the quality tolerance.

15. The method of claim 11, further comprising filtering, using an initial signal processing module and a 12-lead database, high frequencies from the physiological signal using a low pass filter.

16. The method of claim 15, wherein the low pass filter removes a predictable noise element.

17. (canceled)

18. The method of claim 11, wherein the signal characteristic model comprises a statistical model.

19. The method of claim 18, wherein the statistical model comprises a machine learning model.

20. The method of claim 11, wherein the method further comprises conditionally displaying, using the processor and the graphical user interface, a notification as a function of the signal characteristic profile.
